(19) United States
(12) Patent Application Publication
(10) Pub. No.: US 2011/0086097 A1
(43) Pub. Date: Apr. 14, 2011

(54) Manufacture process for the preparation of an iron containing phosphate adsorbent

(76) Inventors: Daniel Kaufmann, Battwil (CH); Nicole Bieri, Muttenz (CH); Ulrich Meier, Riehen (CH); Ranjit Thakur, Basel (CH); Zdenek Zencak, Basel (CH); Christa Hartmann, Zurich (CH); Andreas Meyer, Nuenburg (DE)

(21) Appl. No.: 12/997,447

(22) PCT Filed: Jun. 12, 2009
(86) PCT No.: PCT/EP2009/057307

§ 371 Date: Dec. 10, 2010

(30) Foreign Application Priority Data
Jun. 13, 2008 (EP) 08158263.7

(51) Int. Cl. A61K 33/42; A61K 9/28; A61P 9/00; A61P 5/00; A61P 13/12

(52) U.S. Cl. 424/474; 424/604; 264/13

(57) ABSTRACT

The present invention relates to a new manufacture process for producing an iron containing phosphate adsorbent, in particular to a process for manufacturing and isolating an iron(III)-based phosphate adsorbent which exhibits valuable pharmacological properties.

The present invention relates to a new process for the manufacture of an iron containing phosphate adsorbent, uses thereof and pharmaceutical compositions containing it.

The present invention provides a manufacture process for producing an iron(III) based phosphate adsorbent. In particular there is provided a process for manufacturing and isolating the iron(III) based phosphate adsorbent in a form which is easily packaged, e.g. as a dry powder suitable for direct sachet filling.

Phosphorus is critical for bone mineralization, cellular structure, genetic coding, and energy metabolism. Many organic and inorganic forms exist. Phosphorus is present in nearly all foods, and GI absorption of dietary forms is very efficient. Phosphorus homeostasis normally is maintained through several mechanisms (renal excretion, cellular release, hormonal control, etc). When the phosphorus load (from GI absorption, exogenous administration, or cellular release) exceeds renal excretion and tissue uptake, hyperphosphatemia occurs.

Due to the high levels of phosphate contained in diet and the relatively low adsorption capacity of the phosphate adsorbents available or described in the prior art, it is necessary to administer such adsorbents in high dose in order to efficiently control the blood level of phosphate. Therefore there is a need to provide a phosphate adsorbent with a high phosphate binding capacity to be usable as a pharmaceutical.

Furthermore there is a need to provide a phosphate adsorbent which is characterized by a diminished release and absorption of iron under physiological conditions. Furthermore there is a need to provide a manufacture process which leads to an adsorbent which is homogenous and stable, and which can be easily formulated and/or packaged, and which can be performed on a large scaling without affecting the properties of the adsorbent, i.e. its phosphate binding capacity.

Surprisingly it has been found that by using dedicated reaction conditions during the manufacture process of a polynuclear iron it is possible to prepare a polynuclear iron(III)-based phosphate adsorbent which has a higher phosphate binding capacity than the phosphate adsorbents of the prior art, in particular than the iron-based phosphate adsorbent described in the prior art.

In order to obtain an iron-based compound which can be used as a pharmaceutical, it is necessary to have a manufacture process leading to a product with reproducible high phosphate binding capacity. This need must particularly be fulfilled in case of large scaling up. Surprisingly, it has been found that it is possible to prepare large amounts of a homogeneous iron(III) based phosphate adsorbent, e.g. from several grams up to technical scale thereof, by using adequate reaction conditions, i.e., by using a stabilizer during the thermal stress, such as sucrose, and/or by isolating the product by a mild method such as spray drying or fluidized bed spray drying.

According to the present invention, pharmaceutical composition of the invention refers to pharmaceutical composition containing an iron (III)-based phosphate adsorbent according to the invention.

The present invention includes a process for the preparation of a composition, which process comprises the steps of:
(i) mixing, e.g. simultaneously mixing, an aqueous solution of iron(III) salt with a base, e.g. an aqueous base, to form a suspension with a pH of between 3 and 10, e.g. between 4 and 9, e.g. between 6 and 8, preferably about 7; allowing the suspension to stand;
(ii) isolating the precipitate formed, and optionally washing, e.g. with water;
(iii) suspending the precipitate, e.g. in water to obtain a suspension with an iron content of about 3 to 16% by weight of the suspension;
(iv) adding one or more carbohydrates and/or humic acid to obtain a suspension with an iron content of the isolated solid of about 10 to about 50% by weight of iron; and
(v) isolating the resulting phosphate adsorbent by filtration, decantation, spray drying or fluidized bed spray drying, preferably spray drying or fluidized bed spray drying, most preferred fluidized bed spray drying.

In step (i) the aqueous solution of iron(III) salt with the aqueous base leads first to nucleation and then to precipitation of the iron oxide hydroxide. The nucleation may be performed in presence of an insoluble carbohydrate, e.g. starch, or the carbohydrate may be added after the nucleation, and before the precipitation.

In one embodiment of the invention, the aqueous solution of iron(III) salt is mixed to the aqueous base in the presence of an insoluble carbohydrate, e.g. starch. Optionally, additional insoluble carbohydrate is then added. In another embodiment of the invention, insoluble carbohydrate is added only after having mixed the aqueous base with the iron salt, for example after the precipitation of the iron salt has started.

The iron salt may be iron(III) chloride, iron(III) nitrate or iron(III) sulfate, preferably the iron salt is iron chloride, e.g. solid iron(III)chloride hexahydrate.

The aqueous solution of iron(III) salt may be in particular a solution of iron(III) salt, as herein above defined, in water. The solution of iron salt may comprise from about 3 to about 35 wt/wt %, e.g. about 3 to about 25 wt/wt % of iron salt, preferably about 3 to about 16 wt/wt % of iron salt, based on the total weight of the iron salt. Preferably, a solution of iron(III) chloride at about 3 to about 35 wt/wt %, e.g. about 3 to about 25 wt/wt % of iron salt, preferably about 3 to about 16 wt/wt % based on the total weight of the iron salt, is used.

The base to be used may be hydroxide or carbonates of alkali or alkaline earth metals. Alkali carbonates, alkali bicarbonates and alkali metal hydroxides (e.g. of sodium) are preferred. In particular, the base may be selected from LION, KOH, NaOH, NaHCO₃ Na₂CO₃, Ca(OH)₂, Mg(OH)₂, Li₂CO₃, K₂CO₃, CaCO₃, MgCO₃, preferably Na₂CO₃. The base solution may comprise about 20 to about 30, e.g. about 22 to about 27, e.g. about 25.5 vol % of base, based on the total volume of the solution.

The aqueous base may consist of an aqueous solution containing a base as hereinabove defined.

The amount of base is chosen in order to obtain the desired pH, e.g. to adjust the pH of the solution resulting from the mixture with the aqueous solution of iron(III) salt to a pH between about 3 and about 10, preferably between about 6 and about 8, more preferably about 7.

In a preferred embodiment of the invention, in step (i) the pH of the solution is maintained constant at a pH between about 6 and about 8, preferably about 7 during all the mixing. By simultaneously addition of the iron salt and the base, the pH can be adjusted to and maintained at the desired value throughout the process.

According to the invention, the reaction, in particular step (i), is preferably made at a temperature between about 1 and about 20, preferably between about 2 and about 10, preferably about 5° C. In another embodiment step (i) is performed at ambient temperature.

According to the invention, the precipitate obtained in step (i) may be washed, at least one time.

According to the invention, in step (ii) the obtained precipitate is isolated, e.g. by decantation, filtration, centrifugation, preferably by decantation, and then washed. The washing is performed with water or an aqueous solution of NaHCO₃, preferably with water. Combinations of water washings and NaHCO₃-washings may be used. The precipitate is washed once or several times, preferably several times. Washing can be done until the level of impurities is down to a predefined level. Preferably 2 to 5 washings are done, more preferably 3 to 5. After each washing operation the water or washing solution is removed by decantation, filtration, centrifugation, preferably by decantation. Preferably, the product is not completely dried.

The product is then resuspended in water. A minimum amount of water is needed so that the suspension can be processed. For example the ratio amount of water/final phosphate adsorbent may be from about 0.8 to about 2, preferably 1.1 to 1.5, more preferably about 1.

The resulting aqueous suspension of phosphate adsorbent has approximately an iron content of about 2 to about 16% by weight, preferably of about 3 to about 8%, and preferably a pH value in the range of about 6 to 8.

Subsequently to step (ii) the suspension may stagnate for some time, e.g. more than 1 hour, preferably during 1 to 5 hours. During that time, the suspension can be stirred.

According to the invention, in step (iv) the carbohydrate comprises soluble or insoluble carbohydrate or mixture thereof.

According to the invention, the soluble carbohydrate may be a glucose derivative. Glucose derivatives may be selected from agarose, dextran, dextrin, dextran derivatives, cellulose, cellulose derivatives, sucrose, maltose, lactose, mannitol and mixture thereof. Preferred glucose derivatives are sucrose, maltodextrin and mixture thereof. Most preferred glucose derivative is sucrose.

According to the invention, the amount of soluble carbohydrate, e.g. glucose derivative, added in step iv) may be of about 5 to about 15 weight %, preferably about 5 to about 10 weight %, based on the weight of the phosphate adsorbent. Preferably about 5 to about 15 weight % sucrose or about 5 to about 10 weight % sucrose is used.

According to the invention, the insoluble carbohydrate may be starch. Starch may be selected from corn, wheat, rice, maize, pea or potato starch, and mixture thereof. Starch may also contain part of soluble starch (e.g. maltodextrin). For example, starch may be a mixture of 80 weight % or more of potato starch and 20 weight % or less of soluble starch, e.g. 80 weight % or more of potato starch and 20 weight % or less of maltodextrin. In another embodiment of the invention, the starch is replaced by a dietary fiber, e.g. Benefiber® (produced by Novartis AG). Preferably starch is potato starch.

Preferably e.g. 1 g of insoluble carbohydrate, e.g. starch, is added per about 0.5 to about 30 g of iron salt, e.g. per about 1.0 to about 20 g of iron salt, e.g. per about 1.5 to about 10 g of iron salt, e.g. per about 2.0 to about 15 g of iron salt.

In step (iv), a preservative may be added, e.g. a soluble preservative, such as e.g. chlorhexidine or p-hydroxy-benzoic acid ester, or an alcohol, such as e.g. ethanol, methanol, 2-propanol or combination thereof. Preferably, the preservative is an alcohol. Preferred alcohol is ethanol.

The step (v) consists of isolating the phosphate adsorbent. Such an isolation may be made by filtration, decantation, spray drying or fluidized bed spray drying. Preferably spray drying or fluidized bed spray drying, e.g. fluidized bed spray drying is performed. Unexpectedly, it was found that such a technique leads to produce a well granulated, free flowing and dust free powder which is suitable for direct sachet filling, without the use of excipients, and whose storage stability is significantly superior. That powder can easily be manipulated, e.g. during processing or packaging.

According to the invention, there is provided a process for manufacturing an iron(III)-based adsorbent having high phosphate binding capacity in form of a dry powder, as hereinabove described, wherein the process further comprises a step of isolating the product by spray drying or fluidized bed spray drying. Fluidized bed spray drying is preferred, e.g. NIRO PSD-4, using appropriate process parameters.

Surprisingly, it has been found that fluidized bed spray drying is particularly suitable to directly and continuously produce a well granulated, free flowing and dust free powder which is suitable for direct sachet filling or can be easily granulated to yield a granulate.

The invention discloses a process for the preparation of composition which process comprises the steps of:
(i) mixing an aqueous solution of iron(III) salt with at least sodium carbonate to form a suspension with a pH of between 3 and 10,
(ii) isolating a precipitate formed,
(iii) suspending the precipitate in an aqueous solution,
(iv) adding starch and sucrose, and
(v) isolating the preparation of step (iv) by spray drying or fluidized bed spray drying.

Furthermore a formulation step may be performed following step (v). For example mixing, granulating, encapsulating and/or tabletting the phosphate adsorbent may be done, with adequate excipients if necessary.

According to the invention, there is provided an iron-based phosphate adsorbent comprising carbohydrate and/or humic acid; e.g. soluble or insoluble carbohydrate or mixture thereof. Examples of soluble carbohydrates include sucrose, maltodextrin, agarose, dextran, dextrin, cellulose, maltose, lactose, mannitol or mixture thereof. Preferred soluble carbohydrate is sucrose. Examples of non soluble carbohydrates include starch, agarose, dextran, dextrin, cellulose. Preferred non soluble carbohydrate is starch.

One or more calcium salts, such as calcium acetate, may be added, Examples of suitable calcium salts include salts of inorganic or organic acids, particularly calcium acetate.

In one embodiment of the invention, the iron-based phosphate adsorbent may be defined as a complex formed with polynuclear iron(III) oxide hydroxides, starch and the glucose derivative (e.g. sucrose or maltodextrin, preferably sucrose). In a preferred embodiment, the polynuclear iron oxide hydroxides is bound to the adsorbent base material, e.g. starch.

In one embodiment of the invention, the compound of the invention comprises starch particles covered by iron(III) oxide-hydroxide and optionally stabilized by a water-soluble carbohydrate.

In yet a further aspect of the invention there is provided a new phosphate adsorber containing iron (III) oxide-hydroxide, which contains at least about 20 weight % of iron, for example at least about 25 weight %, for example about 30 weight % of iron, based on the total weight of the product. In another aspect of the invention the iron content of the compound of the invention is about 20 to about 50 weight %, for example about 40 to about 50 weight %, based on the total weight of the product.

Preferably the phosphate adsorbent of the invention comprises iron(III), optionally mixed to ferrihydrite. In a preferred embodiment, the polynuclear iron oxide hydroxide of the phosphate adsorbent consists of gamma-iron or beta-iron oxide hydroxide, preferably beta-iron oxide hydroxide or mixture thereof with ferrihydrite.

In another embodiment of the invention, the polynuclear iron oxide hydroxide of the phosphate adsorbent is x-ray amorphous.

The iron(III)-based phosphate adsorbent according to the invention is, therefore, useful in the treatment and/or prevention of hyperphosphataemia, hypercalcaemia, hyperparathyroidism reduction, in cardiovascular morbidity and mortality, renal osteodystrophy, calciphylaxis and soft tissue calcifications. In particular the iron(III)-based phosphate adsorbent according to the invention is suitable for the treatment and/or prevention of hyperphosphataemia, in humans and warm-blooded animals, in particular companion animals such as dogs and in particular cat.

The phosphate adsorbent of the invention, and pharmaceutical composition containing it are more particularly useful in patients with hyperphosphataemia, e.g. for dialysis-dependent patients, e.g. hemodialysis, or patients suffering from advanced chronic kidney diseases (CKD), chronic renal failure, chronic renal insufficiency, end-stage renal disease.

The phosphate adsorbent of the invention, and pharmaceutical composition containing it are more particularly useful for controlling serum phosphate and serum calcium-phosphate product levels, while maintaining normal serum calcium levels, in a subject in need of such treatment; e.g. in patients on chronic hemodialysis, by administering to said subject an effective amount of an iron(III)-based phosphate adsorbent according to the invention.

In another embodiment of the invention, the phosphate adsorbent of the invention, and pharmaceutical compositions containing it are also useful for selectively removing inorganic phosphate or eliminating inorganic phosphate from dialysis fluids, whole blood or plasma; e.g. in patients on dialysis, e.g. on chronic hemodialysis, by administering to said subject an effective amount of an iron(III)-based phosphate adsorbent according to the invention.

Pharmaceutical compositions according to the invention may be formulated in any conventional form, preferably oral dosage forms, e.g. powders, granules, granulates, capsules, sachets, stick packs, bottles (optionally together with adequate dosing systems, e.g., calibrated spoons), tablets, dispersible tablets, film coated tablets, or uniquely coated tablets.

Pharmaceutical compositions according to the invention may also be formulated as semi-solid formulations, e.g. aqueous and non aqueous gel, swallowable gel, chewy bar, fast-dispersing dosage, cream ball chewable dosage form, chewable dosage forms, or edible sachet as defined herein below.

Preferred formulations are powder, granulate, tablet, for example dispersible tablet.

In a preferred embodiment of the invention, the pharmaceutical composition is prepared in the form of a powder or a granulated product (i.e. granulated powder or granulates), which is optionally filled into powder containers such as bottle, capsule, sachet or stick pack. Optionally such a sachet or stick pack is supplied with a child resistant easy opening feature. A lubricant, as defined herein below, may be added, for example in case the phosphate adsorbent of the invention, e.g. as prepared according to the manufacture process defined hereinabove, is filled into a capsule.

The granulated product may be prepared by dry granulation, e.g. roller compaction, or wet granulation, for example in a fluid bed or high shear mixer. The granulation may be done in presence of a binder, e.g., MCC, in order to improve the mechanical stability of the granulate. The granulate may be filled then into e.g. bottles, capsules, sachets or stick packs. In one embodiment of the invention such filling can be performed by automatically working systems. The sachet or stick packs may contain between about 0.5 to 10 g, e.g. from about 0.5 to 5 g of granulated product.

The pharmaceutical composition of the invention may contain a binder, e.g. dry binder, such as sucrose or microcrystalline cellulose (MCC).

In another embodiment of the invention, the pharmaceutical composition of the invention may contain a lubricant, e.g. Mg-stearate or hydrophilic lubricant, such as PEG 6000 or PEG 4000. The invention provides a capsule containing the iron(III)-based phosphate adsorbent, e.g. as a powder or a granulate, and preferably further comprising a lubricant.

According to one embodiment of the invention, the pharmaceutical composition is in form of a tablet. For better applicability or ease of differentiation, a subsequent film coating of the tablet may be performed.

The tablet may be produced by tabletting, e.g. direct compressing, the phosphate adsorbent as a pure powder, i.e. without containing any excipient.

In another embodiment of the invention, the tablet is prepared by compression of the pure powder, i.e. a powder of the phosphate adsorbent without excipient, together with suitable excipients, such as excipients selected from filler, binder, disintegrant, flow agent, lubricant, and mixture thereof.

In yet another embodiment of the invention, the tablet is obtained by compression of the granulated powders (i.e. the “inner phase”) together with further excipients (the “outer phase”). The inner phase of the pharmaceutical composition according to the invention may comprise the phosphate adsorbent, and at least one excipient selected from a filler, a binder, a disintegrant, and mixture thereof. The outer phase of the pharmaceutical composition according to the invention may comprise at least one excipient selected from a flow agent, a lubricant, a filler, a disintegrant and mixture thereof. Preferably the outer phase comprises a flow agent, a lubricant, and optionally a filler and/or a disintegrant.

The pharmaceutical compositions according to the present invention may comprise a filler to provide processability.

Suitable filler materials are well-known to the art (see, e.g., Remington's Pharmaceutical Sciences, 18th Ed. (1990), Mack Publishing Co., Easton, Pa., pp. 1635-1636), and include microcrystalline cellulose, lactose and other carbohydrates, starch, pregelatinized starch, e.g., starch 1500R (Colorcon Corp.), corn starch, dicalcium phosphate, potassium bicarbonate, sodium bicarbonate, cellulose, calcium phosphate dibasic anhydrous, sugars, sodium chloride, and mixtures thereof, of which lactose, micro-crystalline cellulose, pregelatinized starch, and mixtures thereof, are preferred. Owing to its superior disintegration and compression properties, microcrystalline cellulose (Avicel grades, FMC Corp.), and mixtures comprising microcrystalline cellulose and one or more additional fillers, e.g., corn starch or pregelatinized starch, are particularly useful. Preferably the filler is microcrystalline cellulose.

The filler may be present in an amount of about 10 to 40 weight %, based on the total weight of the pharmaceutical composition, preferably 20 to 40 weight %, more preferably about 30 weight %.

The pharmaceutical composition of the invention may contain also the following classes of excipients:
a) well-known tabletting binders (e.g., hydroxypropylmethylcellulose, starch, starch pregelatinized (starch 1500), gelatin, sugars, natural and synthetic gums, such as carboxymethyl-cellulose, methylcellulose, polyvinylpyrrolidone, low substituted hydroxypropylcellulose, ethylcellulose, polyvinylacetate, polyacrylates, gelatin, natural and synthetic gums), micro-crystalline cellulose, and mixtures of the foregoing. In a preferred embodiment the binder consists of low substituted hydroxypropylcellulose HPC (e.g. HP cellulose-LH22) or hydroxypropylmethylcellulose HPMC, e.g. 3 or 6 cps.

The tabletting binder may be comprised between about 1 and about 10 weight %, and preferably about 1 and about 5 weight %, based on the total weight of the pharmaceutical composition. In a preferred embodiment, the binder is used at about 3 weight %, based on the total weight of the pharmaceutical composition.
b) disintegrants, e.g. carboxymethylcellulose, cross-linked sodium carboxymethyl-cellulose (croscarmelose sodium), crospovidone, sodium starch glycolate. Preferred disintegrants are crospovidone and croscarmelose sodium.

The disintegrant may be comprised between about 3 and about 15 weight %, preferably about 5 and about 10 weight %, based on the total weight of the pharmaceutical composition. For example, the disintegrant is crospovidone, croscarmelose sodium or mixture thereof, and is contained at about 10 weight %, based on the total weight of the pharmaceutical composition.
c) lubricants, e.g. magnesium stearate, stearic acid, calcium stearate, glyceryl behenate, hydrogenated vegetable oil, carnauba wax and the like, polyethylene oxides such as PEG 6000 or PEG 4000. In a preferred embodiment lubricant is magnesium stearate.

The lubricant, e.g. magnesium stearate, may be present from about 0.5 to about 5 wt ° A), e.g. from about 3 to about 5 weight %, preferably about 2 to about 3 weight %, based on the total weight of the pharmaceutical composition.
d) flow agents, e.g. silicon dioxide or talc, preferably silicon dioxide colloidal (e.g. Aerosil). The flow agent, e.g. silicon dioxide colloidal, may be present from about at about 0.1-2 weight %, e.g. 0.5 weight %, based on the total weight of the pharmaceutical composition.
e) anti-adherents or glidants, e.g., talc;
f) sweeteners;
g) opacifying or coloring mediums, e.g. titanium dioxide, iron oxide or aluminum lakes;
h) flavoring mediums;
i) antioxidants.

According to the invention, there is provided a tablet containing the iron(III)-based phosphate adsorbent, and a lubricant, and optionally at least one further excipient selected from a filler, a binder, a disintegrant, and a flow agent, as hereinabove described. The tablet may further comprise at least one excipient selected from an anti-adherent, a glidant, a sweetener, an opacifying or coloring medium, and a flavoring medium, as hereinabove described.

The tablet may be coated, e.g. may comprise a film coating. Examples of suitable film formers in film coating compositions to be applied to pharmaceutical compositions of the invention comprise e.g. polyethylene glycol, polyvinylpyrrolidone, polyvinyl alcohol, hydrophilic polymers such as cationic polymers containing dimethylamino-ethyl methacrylate as functional groups (e.g. Eudragit E and EPO), hydroxypropylcellulose, hydroxymethylcellulose, and hydroxypropylmethylcellulose or the like, of which hydroxypropylmethylcellulose is preferred.

The film coating composition ingredients include plasticizers, e.g. polyethylene glycols (e.g. polyethylene glycol 6000), triethylcitrate, diethyl phthalate, propylene glycol, glycerin in conventional amounts, as well as the above-mentioned opacifiers such as titanium dioxide, and colorants, e.g. iron oxide, aluminum lakes, etc. Preferably dry mixtures as Sepifilm or Opadry mixtures, latter prepared by Colorcon Corp. are used. These products may be individually prepared dry pre-mixtures of film forming polymers, opacifiers, colorants and plasticizers which are further processed to aqueous film coating suspensions.

The film coating may be generally applied to achieve a weight increase of the tablet of about 1 to 10 weight %, and preferably about 2 to 6 weight %, based on the total weight of the pharmaceutical composition.

The film coating can be applied by conventional techniques in a suitable coating pan or fluidized bed apparatus using water and/or conventional organic solvents (e.g., methyl alcohol, ethyl alcohol, isopropyl alcohol), ketones (acetone), etc.

In another embodiment of the invention, the iron(III)-based phosphate adsorbent is formulated as an uniquely coated tablet.

The tablet according to the invention may be made by direct compression of the iron(III)-based phosphate (the drug substance) and by addition of high concentrations of Mg-stearate (e.g. about 3 to about 5%).

The tablet may further comprise binders as e.g. HPMC 3cPs, HP-Cellulose LH-22.

Electrostatic dry powder deposition process may increase the structural integrity of the tablet without adding major amounts of material and also provides the opportunity for an unique appearance of the dosage form.

The tablet may be coated by a electrostatic dry powder deposition process, e.g. as follows:

The coating mixture is prepared by melt-extrusion of a mixture of polymer (preferably Eudragits, e.g. type E, RS. L, RL and additionally PVP/VA, HPMPC, HPMCAS), coloring agent (e.g. titan dioxide) and other additives (e.g. PEG3000). A further step of micronization of the produced melt-extrudate is optionally performed, e.g. with about 7 to 10 micron. The coating process may consist of i) fixing the core (e.g. by vacuum) on a wheel, charged, transported through the coating chamber and attaching the opposite charged coating powder to the core surface, ii) transporting the powder layered core on the wheel to an IR lamp were the coat melts, iii) transferring the core to the adjacent second wheel and repeating the process for the bottom part of the tablet core. Typical coat weights are 3-4% of the core weight and are about 20-50 μm thick.

Heat fixation step: The fusion cycle varies from product to product but typically is around 80s per side. This includes heating up the tablets from room temperature, so the temperature at the surface of the tablet peaks at approximately 100° C. and in the tablet core approximately 70° C. for about 20 s.

According to the invention, the iron(III)-based phosphate adsorbent according to the invention may also be formulated as semi solid formulations. Such compositions are comfortable to swallow, in particular for elderly and children, and may be considered as a daily supplement rather than a medicine. Furthermore such semi-solid dosage forms have the advantage that they may be filled into multiple or single dose containers.

In one embodiment of the invention, the composition of the invention is in form of an aqueous gel formulation. Such an aqueous gel may contain a viscosity enhancer which preferably has wetting properties, or a thickener. The viscosity may be selected from polyoxyethylene sorbitan fatty acid esters, polyethylene glycols, and glycerol. The thickener may be selected from starch (e.g. corn starch, potato starch, pea starch), the starch being preferably heated up, cellulose derivative (e.g. hydroxypropylmethyl cellulose), alginate salt (e.g. sodium alginate), carbomer, colloidal silicon dioxide, and other paste former (such as e.g. PVP, polyacrylic acid, arabic gum, xanthan gum and mixture thereof).

In addition, preservatives may be added, such as e.g. p-hydroxybenzoic acid methyl ester and salt thereof, p-hydroxyl benzoic acid propylester and salt thereof, sorbic acid and salt thereof, benzoic acid and salt thereof or chlorhexidine. Flavours and sweeteners may also be added. The aqueous gel may contain a buffering system, e.g. citrate or acetate buffer, to ensure the antimicrobial efficacy of the preservative system.

The aqueous gel may further contain at least one agent selected from a sweetener, such as e.g. saccharin sodium, aspartame, sucralose and a flavour, such e.g. as strawberry or passion.

The aqueous gel may be prepared by solubilizing all excipients, with the exception of the thickener in water purified, dispersing the phosphate adsorbant until vigorous mixing and then by adding the thickener.

Possible semi solid formulations include, but are not limited to, swallowable gel, e.g. aqueous or non aqueous gel (the phosphate adsorbent being optionally encapsulated or granulated); chewy bar, e.g. a cereal bar; fast-dispersing dosage, such as orally dispersing wafer; cream ball chewable dosage form; chewable dosage forms, such as candy, soft capsule, or nugget; or edible sachet. In such semi solid formulations, the iron(III)-based phosphate adsorbent may contain dietary fiber as insoluble carbohydrate, e.g. the starch may be replaced by dietary fibers.

Semi solid formulations have the advantage that they may be perceived as a daily supplement rather than a medicine, which means that a rather large dosage form may be acceptable for the patients. Preferably, these formulations are given to elderly and pediatric patients.

Swallowable gel has the advantage of be comfortable to swallow and be possibly perceived as a daily supplement rather than a medicine. In addition there is a wide choice of flavor options. Non-aqueous gels are preferred. Encapsulation of the iron hydroxide and/or a granulation step is preferably included, in order to overcome problems with mouth feeling, e.g. grittyness.

According to the invention, the chewy bar may contain ingredients selected from the groups consisting of malt extract, skimmed milk powder, fat reduced cacao, glucose syrup, egg, hardened palm oil (e.g. about 30 weight %, based on the total weight of the bar), yeast, sodium chloride (e.g. about 0.1 weight %, based on the total weight of the bar), vitamin (e.g. vitamin E), favor (e.g. vanilla flavor), one or more stabilizers (e.g. E339, E435, E472b, E475, soy bean lecithin), thickener (e.g. carob flour, E460). The bar may be covered by a milk chocolate layer, e.g. containing sugar, cacao, cacao butter, whole milk, skimmed milk powder, hazelnuts, butterfat, soy bean lecithin. The cover may weight 33% of the total weight of the chewy bar. The manufacturing process may comprise blending of all the ingredients in a mixer at elevated temperature and filling the blend into a mold. The bars may be packaged after cooling to room temperature and removal from the mold.

Chewing of a chewy bar, e.g. a cereal bar is a convenient and patient friendly administration and may be perceived as part of the daily routine, i.e. daily supplement rather than a medicine. Such a dosage form has only minor limitations with respect to the size. In addition there is a wide choice of flavor options.

Orally dispersing wafers are versatile fast-dispersing dosage form. Orally dispersing wafers containing the phosphate adsorbent of the invention, e.g. iron(III)-based phosphate are particularly suited for pediatric and geriatric populations, since they are comfortable to swallow and may be perceived as a daily supplement rather than a medicine.

According to the invention, a rapidly dispersing dosage form may release its active ingredient, i.e. the iron(III)-based phosphate adsorbent, within a period of less than about ninety seconds. These dosage forms may exhibit a three-dimensional shape, which can be retained for adequate storage but may be readily dispersed in the presence of excess moisture.

According to the invention, the rapidly dispersing dosage, e.g. the orally dispersing wafer, may be manufactured by a solid, free-form fabrication technique in which objects are built in a laminated fashion through sequential addition of patterned thin layers, e.g. three dimensional printing (3DP).

According to the invention the semi-solid dosage may be a cream ball chewable dosage form. In one embodiment of the invention, the phosphate adsorbent is suspended in a cream or gel and then layered on a core. Various flavors may be used. Such a form may provide better chewability and mouth feeling than other chewable dosage forms. This formulation may be comfortable to swallow and may be perceived as a daily supplement rather than a medicine.

According to the invention, chewable dosage forms include for example candy, soft capsule, and nugget. A wide choice of flavor may be used. Fancy shapes and colors can be designed. The chewable dosage form may be packed in a tablet dispenser or individually wrapped.

According to the invention, the chewable dosage form may contain ingredients selected from the group consisting of: corn syrup, sugar, partially hydrogenated soybean and cottonseed oil, nonfat milk powder, soy lecithin, natural or artificial flavor, citric acid, glyceryl monostearate, Carrageenan, Red 40, Vitamin (e.g. Vitamin D3 or K1), tricalcium phosphate, alpha tocopheryl, salt, niacinamide, calcium pantothenate, pyridoxine hydrochloride, riboflavin, and thiamine mononitrate.

The ingredients may be dissolved in water or in milk to form a syrup, which may be boiled until it reaches the desired concentration or the sugar starts to caramelize. The liquid may then be filled onto molds and cooled down to harden the dosage form.

According to the invention, the phosphate adsorbent, may be formulated as an edible sachet. Eating a sachet is a convenient and patient friendly administration and may be perceived as part of the daily routine, i.e. as a daily supplement rather than a medicine.

The filling of the edible sachet may be made e.g. consist of, of granules, which may be made of the material as hereinabove described for the chewy bars. For example, the filling of the edible sachet may be made by milling of the bars after removal from the mold. The sachet material may be made of water soluble polysaccharide, e.g. starch, mashed vegetable or fruits, optionally with lipids. The sachet may be manufactured by spraying the fruit or vegetable puree on a fast rotating teflonized disk where it forms a thin film which is dried in a next step.

In another embodiment of the invention, the non soluble carbohydrate contained in the iron(III)-based phosphate adsorbent is a dietary fiber, e.g. Benefiber®. For example, in step i) and/or step ii) of the manufacture process as described hereinabove, starch is replaced by dietary fiber, e.g. Benefiber®. Such a formulation combines the benefits of phosphate binding and of dietary fibers in one product.

The iron(III)-based phosphate adsorbent according to the invention exhibit valuable pharmacological properties, e.g. adsorbing inorganic phosphate or phosphate bound to foodstuffs from body fluids or foodstuffs, e.g. as indicated in in vitro and in vivo tests and are therefore indicated for therapy.

The iron(III)-based phosphate adsorbent according to the invention is therefore, useful in the treatment and/or prevention of hyperphosphataemia, hypercalcaemia, hyperparathyroidism reduction, in cardiovascular morbidity and mortality, renal osteodystrophy, calciphylaxis and soft tissue calcifications. In particular the iron(III)-based he phosphate adsorbent according to the invention is suitable for the treatment and/or prevention of hyperphosphataemia, in humans and warm-blooded animals, in particular companion animals such as dogs and in particular cat

The phosphate adsorbent of the invention, and pharmaceutical compositions containing it are more particularly useful in patients with hyperphosphataemia, e.g. for dialysis-dependent patients, e.g. hemodialysis, or patients suffering from advanced chronic kidney diseases (CKD), chronic renal failure, chronic renal insufficiency, end-stage renal disease.

The phosphate adsorbent according to the invention may be administered by any conventional route, in particular enterally, e.g. orally, e.g. in the form of tablets or capsules. In some cases the phosphate adsorbent may be administered through nasogastric tubes, e.g. pediatric naso-gastric tubes.

Pharmaceutical compositions comprising the compound of the invention in association with at least one pharmaceutical acceptable carrier or diluent may be manufactured in conventional manner by mixing with a pharmaceutically acceptable carrier or diluent.

Unit dosage forms for oral administration contain, for example, from about 0.5 g to about 7 g, e.g. from about 0.5 to about 5 g, e.g. from about 1.0 to about 3 g, preferably from about 1 to about 1.5, more preferably about 1 to about 1.5 g, even more preferably about 1 to about 1.25 g of phosphate adsorbent.

The phosphate adsorbent according to the invention may also be used for the absorption of phosphate bound to foodstuffs. They may be admixed with foodstuffs.

Utility of the iron(III)-based phosphate adsorbent of the invention, in the treatment of hyperphosphateamia, may be demonstrated in animal test methods as well as in clinic, for example in accordance with the methods hereinafter described.

A—Phosphate Binding capacity may be determined in assay done according to published methods, e.g. as described in WO2007/088343, the content thereof being incorporated by reference, or according to present Example 2.
B—Clinical Trial: open label, time-lagged, multiple dose, switch study in patients with CKD (chronic kidney diseases) on hemodialysis.

Patients remain on their current sevelamer treatment during a 2-week run-in period, then enter a 1- to 2-week wash out period prior to being switched to the iron(III)-based phosphate adsorbent as described in Example 1 for 4 weeks: 3.75 g/day, 7.5 g/day, 11.25 g/day, 15 g/day, 22.5 g/day. Each cohort enrolls 10 patients. Patients are stratified by pre-study sevelamer dose: Strata 1 is less than 7.2 g/day of sevelamer in cohorts of 3.75 g/day and 7.5 g/day of the iron(III)-based phosphate adsorbent. Strata 2 is greater or equal to 7.2 g/day of sevelamer in other cohorts of the iron(III)-based phosphate adsorbent treatment.

In accordance with the foregoing the present invention provides:
1.1 An iron(III)-based phosphate adsorbent characterized by an improved phosphate binding capacity. The binding capacity is of at least about 50 mg adsorbed phosphate by 1 g of phosphate adsorbent, preferably of about 120 mg adsorbed phosphate by 1 g of phosphate adsorbent, most preferred of about 140 mg adsorbed phosphate by 1 g of phosphate adsorbent, even most preferred about 200 mg adsorbed phosphate by 1 g of phosphate adsorbent.
1.2 An iron(III)-based polynuclear iron(III)-based phosphate adsorbent comprising i) polynuclear iron(III) oxide hydroxides, ii) an adsorbent base material, preferably a non soluble carbohydrate, and iii) a soluble carbohydrate, e.g. glucose derivative, and iv) optionally a carbonate, wherein the soluble carbohydrate is partially incorporated into the polynuclear iron (III) oxide hydroxides.
1.3 A polynuclear iron(III)-based phosphate adsorbent comprising i) polynuclear oxide hydroxides, and ii) a glucose derivative selected from sucrose, maltodextrin and mixture thereof, preferably sucrose, wherein the glucose derivative is partially incorporated into the polynuclear iron(III) oxide hydroxides, and iii) starch. Optionally the polynuclear iron oxide hydroxides are stabilized by said glucose derivative.
1.4 A polynuclear iron(III)-based phosphate adsorbent comprising i) polynuclear iron(III) oxide hydroxides, and ii) a glucose derivative selected from sucrose, maltodextrin and mixture thereof, preferably sucrose, wherein the polynuclear iron oxide hydroxide contains polynuclear gamma-iron oxide hydroxide, and iii) a non soluble carbohydrate, preferably starch, and optionally ferrihydrite. Optionally the glucose derivative is partially incorporated into the polynuclear iron (III) oxide hydroxides.
1.5 An iron(III)-based phosphate adsorbent comprising i) polynuclear iron (III) oxide hydroxides, ii) an adsorbent base material, preferably a non soluble carbohydrate (e.g. starch), and iii) glucose derivative selected from sucrose, maltodextrin or mixture thereof, preferably sucrose, wherein the polynuclear iron oxide hydroxides are stabilized by said glucose derivative.

In accordance with the foregoing the present invention further provides:
2.1 A process for the preparation of an iron(III)-based phosphate adsorbent containing iron(III) oxide-hydroxide, which process comprises the steps of:
   (i) mixing an aqueous solution of iron(III) salt with at least one base to form a suspension with a pH of between 3 and 10,
   (ii) isolating a precipitate formed,
   (iii) suspending the precipitate in an aqueous solution,
   (iv) adding one or more carbohydrates and/or humic acid, and
   (v) isolating the preparation of step (iv) by spray drying or fluidized bed spray drying.
2.2 A process for the preparation of an iron(III)-based phosphate adsorbent containing iron(III) oxide-hydroxide, insoluble carbohydrate (preferably starch) and a glucose derivative, which process comprises the steps of
   (i) mixing, e.g. simultaneously mixing, an aqueous solution of iron(III) salt with a base, e.g. an aqueous base, to form a suspension with a pH of between 3 and 10, e.g. between 4 and 9, e.g. between 6 and 8, preferably around 7; allowing the suspension to stand,
   (ii) isolating the precipitate formed, and optionally washing, e.g. with water;
   (iii) suspending the precipitate, e.g. in water to obtain a suspension with an iron content of about 3 to 16% by weight of the suspension; and
   (iv) adding one or more carbohydrates and/or humic acid to obtain a suspension with an iron content of up to 10-50% by total weight of the suspended solid; and
   (v) isolating the phosphate adsorbent by filtration, decantation, spray drying or fluidized bed spray drying. Preferably spray drying or fluidized bed spray drying.
2.3 A process for the preparation of an iron(III)-based phosphate adsorbent containing iron(III) oxide-hydroxide, insoluble carbohydrate (preferably starch) and a glucose derivative, which process comprises the steps of
   i) mixing, e.g. simultaneously mixing, an aqueous solution of iron(III) salt with a base, e.g. an aqueous base, to form a suspension with a pH of between 3 and 10;
   ii) adding said insoluble carbohydrate (preferably starch), before the precipitation of the iron(III) is complete, e.g. has started;
   wherein steps iii) to v) are performed as defined under 2.1.
2.4 A process as defined under 2.1 to 2.3 wherein the process further comprises the step of granulating the powder, optionally in presence of at least one excipient selected from a binder and a lubricant, to yield an iron(III)-based phosphate adsorbent as a granulate.
2.5 A process as defined under 2.1 to 2.4 wherein the process further comprises the step viii) of tabletting either the powder obtained in step vi) or the granulate obtained in step vii), wherein the tabletting step is optionally performed in presence of an excipient selected from a filler, a binder, a disintegrant, a flow agent, a lubricant, and mixture thereof, as hereinabove described.

In accordance with the foregoing the present invention further provides:
3.1 A method for preventing or treating disorders or diseases such as indicated above, in a subject in need of such treatment, i.e. a human or a warm-blooded animal, in particular companion animal such as dog and cat, which method comprises administering to said subject an effective amount of an iron(III)-based phosphate adsorbent according to the invention.
3.2 A method for controlling serum phosphate and serum calcium-phosphate product levels, while maintaining normal serum calcium levels, in a subject in need of such treatment, e.g. in patients on chronic hemodialysis, which method comprises administering to said subject an effective amount of an iron(III)-based phosphate adsorbent according to the invention.
3.3 A method for selectively removing inorganic phosphate or eliminating inorganic phosphate, e.g. from dialysis fluids, whole blood, plasma, in a subject in need of such treatment, e.g. in patients on dialysis, e.g. on chronic hemodialysis, which method comprises administering to said subject an effective amount of an iron(III)-based phosphate adsorbent according to the invention.
3.4 A method for selectively removing inorganic phosphate bound to foodstuffs.
3.1 A phosphate adsorbent according to the invention for use as a pharmaceutical, e.g. in any of the methods as indicated under 4.1 to 4.3 above.
4. Composition for use as a pharmaceutical preparation for the selective elimination of inorganic phosphate from liquids, wherein the composition is insoluble in water and contains an iron(III)-based phosphate adsorbent as defined in any preceding claims
5.1 A pharmaceutical composition, e.g. for use in any of the methods as in 3.1 to 3.3 above comprising an iron(III)-based phosphate adsorbent according to the invention in association with a pharmaceutically acceptable diluent or carrier therefore, e.g. comprising at least one excipient selected from a preservative and a binder.
5.2 A pharmaceutical composition, e.g. for use as a pharmaceutical preparation for the selective elimination of inorganic phosphate from liquids, e.g. dialysis fluids, whole blood or plasma, wherein the composition contains an iron(III)-based phosphate adsorbent material according to the invention.
5.3 A pharmaceutical composition suitable for oral administration, e.g. a solid or semi solid dosage form, containing the iron(III)-based phosphate adsorbent according to the invention.
5.4 A solid or semi solid dosage form containing the iron(III)-based phosphate adsorbent according to the invention.
5.5 Pharmaceutical composition, preferably a powder or a granulate, comprising the iron(III)-based phosphate adsorbent according to the invention and further comprising a preservative (e.g. an alcohol, preferably ethanol) and optionally a binder (e.g. sucrose, microcrystalline cellulose or mixture thereof).
5.6 Pharmaceutical composition according to the invention which is in form of a tablet and further comprises a lubricant, and optionally at least one further excipient selected from a filler, a binder, a disintegrant, and a flow agent.
6. An iron(III)-based phosphate adsorbent according to the invention for use in the preparation of a pharmaceutical composition for use in any of the method as in 4.1 to 4.3 above.
6.1 An iron(III)-based phosphate adsorbent according to the invention for use in the treatment or prevention of hyperphosphatemia, hypercalcaemia, hyperparathyroidism reduction, in cardiovascular morbidity and mortality, renal osteodystrophy, calciphylaxis and soft tissue calcifications, and diseases and disorders related thereto.

According to the invention, the phosphate adsorbent may be administered as the sole active ingredient or together with another phosphate reducing agent, such as sevelamer; fosrenol; Ca acetate; or Ca carbonate. It may also be administered in combination with a calcimimetic such as cinacalcet; vitamin D; or calcitriol.

In accordance with the foregoing the present invention provides in a yet further aspect:
7. A method as defined above comprising co-administration, e.g. concomitantly or in sequence, of a therapeutically effective amount of a phosphate adsorbent according to the invention, and a second drug substance, said second drug substance being another Phosphate reducing agent, a calcimimetic, vitamin D, or calcitriol, e.g. as indicated above.
8. A therapeutic combination, e.g. a kit, comprising a) a phosphate adsorbent according to the invention, and b) at least one second agent selected from an another Phosphate reducing agent, a calcimimetic, vitamin D and calcitriol. Component a) and component b) may be used concomitantly or in sequence. The kit may comprise instructions for its administration.

Where a phosphate adsorbent according to the invention is to be administered in conjunction with another phosphate reducing agent, such as sevelamer, fosrenol, Ca acetate or Ca carbonate; a calcimimetic such as cinacalcet; or with vitamin D or calcitriol, e.g. for preventing or treating hyperphosphataemia or other diseases or disorders as hereinabove specified, dosages of the co-administered compound will of course vary depending on the type of co-drug employed, on the condition being treated and so forth.

Pharmaceutical compositions comprising the compound of the invention in association with at least one pharmaceutical acceptable carrier or diluent may be manufactured in conventional manner by mixing with a pharmaceutically acceptable carrier or diluent.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

### EXAMPLES

The following examples are illustrative of the invention.

### Example 1

To a solution of 648 g sodium carbonate dissolved in 2974 g water a solution of 813 g iron(III) chloride hexahydrate dissolved in 3312 g water is added over 34 minutes time span. The resulting suspension is allowed to stand for 2 hours. During this period it is stirred for 10 minutes, six times. The suspension is treated 5 times with 4500 g water with stirring, subsequently allowed to stand for a period of between 1 h and 200 h in order to let the solids settle down. The supernatant liquid was removed by decantation.

225 g saccharose and 225 g starch were subsequently added to the above 3238 g of suspension (LOD analyzed by Halogen Moisture Analyzer HR83=80.83%) resulting in 3688 g of a suspension having a pH of 7.54 at 21.7° C. The iron content of the precipitated solids, suspended in water, is 19.2% (determined by flame Atomic Absorption Spectroscopy or flame AAS). Two qualities of final product are obtained by spray drying (SD) and fluidized bed spray drying (FSD) by using appropriate SD and FSD conditions. The iron content (determined by flame AAS) is 22.1% for the SD product and 21.1% for the FSD product.

### Example 2

The phosphate adsorption of the product obtained in Example 1 is measured by placing 365 to 385 mg of the sample in a 25 mL volumetric flask. The flask is filled with 20 mL of aqueous solution containing 170.92 mg phosphate (PO43-) and the pH is adjusted to pH 2.0 (+/−0.05) with hydrochloric acid. The flask is then filled to mark with water. This sample is then stirred at 37° C. for 2 h. After this, the sample is filtrated and the dissolved phosphate is quantitatively measured by ion chromatography. The amount of adsorbed phosphate is the difference between the amount of phosphate placed in the flask and the measured amount of phosphate. The result is expressed in % (m/m) as: “mass of adsorbed phosphate”/“mass of spray dried product used for adsorption”*100

### Results:

The compound of Example 1 adsorbs 16.1% m/m phosphate for the SD product and 18.7% m/m for the FSD product.

1. A process for the preparation of composition which process comprises the steps of:
(i) mixing an aqueous solution of iron(III) salt with at least one base to form a suspension with having a pH of between 3 and 10,
(ii) isolating a precipitate formed,
(iii) suspending the precipitate in an aqueous solution,
(iv) adding one or more carbohydrates and/or humic acid, and
(v) isolating the preparation of step (iv) by spray drying or fluidized bed spray drying.

2. (canceled)

3. The process according to claim 1 wherein in step (i) the pH is between 6 and 8.

4. The process according to claim 1 wherein in step (i) the pH of the solution is maintained at a constant value between 6 and 8.

5. The process according to claim 1 wherein in step (i) the base is selected from sodium carbonate or sodium bicarbonate.

6. (canceled)

7. The process according to claim 1 wherein in step (iii) the precipitate is suspended in water to form a suspension with an iron content of 3 to 16% by total weight of the suspension.

8. (canceled)

9. The process according to claim 1 wherein in step (iv) the composition contains between 15 and 30% of iron by total weight of the suspended solid.

10. The process according to claim 1 wherein in step (iv) one or more carbohydrates are added.

11. The process according to claim 10 wherein in step (iv) the carbohydrate comprises a soluble or insoluble carbohydrate or a mixture thereof.

12. The process according to claim 10 wherein in step (iv) at least one soluble and at least one insoluble carbohydrates are added.

13. The process according to claim 12 wherein the insoluble carbohydrate is starch.

14. The process according to claim 12 wherein the soluble carbohydrate is selected from the group consisting of sucrose, agarose, dextran, dextrin, dextran derivatives, cellulose, cellulose derivates, maltose, lactose, mannitol and mixture thereof.

15. The process according to claim 1 wherein at least one washing is performed between step (iii) and (iv).

16. The process according to claim 1 wherein in step (i) the pH of the solution is maintained at a value between 6 and 8, and starch is added at step (i).

17. The process according to claim 1 wherein in step (v) isolating the preparation of step (iv) is performed by fluidized bed spray drying.

18. The process according to claim 1 wherein the process further comprises the step (vi) of granulating the powder, optionally in presence of at least one excipient selected from a binder and a lubricant, to yield an iron(III)-based phosphate adsorbent as a granulate.

19. The process according to claim 18 wherein the process further comprises the step (vii) of tabletting either the powder obtained in step (v) or the granulate obtained in step (vi), wherein the tabletting step is optionally performed in presence of an excipient selected from a filler, a binder, a disintegrant, a flow agent, a lubricant, and mixture thereof.

20. An iron(III)-based phosphate adsorbent obtainable by a process according to claim 1.

21. (canceled)

22. A pharmaceutical composition suitable for oral administration comprising the iron(III)-based phosphate adsorbent according to claim 20.

23. A solid dosage form comprising the pharmaceutical composition according to claim 22 which is a powder, a granulate, a capsule, a tablet, or a film-coated tablet.

24. (canceled)

25. (canceled)

26. A method for treating or preventing hyperphosphatemia, hypercalcaemia, hyperparathyroidism reduction, in cardiovascular morbidity and mortality, renal osteodystrophy, calciphylaxis and soft tissue calcifications, and diseases and disorders related thereto in a patient in need thereof, said method comprising administering to the patient an effective amount of the iron(III)-based phosphate adsorbent according to claim 20.
